(19) United States
(12) Patent Application Publication
(10) Pub. No.: US 2023/0146555 A1
(43) Pub. Date: May 11, 2023

(54) ELECTRONIC DEVICE FOR MONITORING VOICE AND LARYNGEAL DISORDERS

(71) Applicant: Electronics and Telecommunications Research Institute, Daejeon (KR)

(72) Inventors: Chan-Hwa HONG, Daejeon (KR); Hye Jin KIM, Daejeon (KR); Saerom SEO, Daejeon (KR); Wooseup YOUM, Daejeon (KR); Yun-Jeong KIM, Daejeon (KR); Hanbit JIN, Daejeon (KR)

(73) Assignee: Electronics and Telecommunications Research Institute, Daejeon (KR)

(21) Appl. No.: 17/901,031

(22) Filed: Sep. 1, 2022

(30) Foreign Application Priority Data
Nov. 10, 2021 (KR) 10-2021-0154106

(51) Int. Cl. A61B 5/11; A61B 5/00

(52) U.S. Cl. CPC A61B 5/1107; A61B 5/6822; A61B 5/4842; A61B 2562/0247; A61B 2562/166

(57) ABSTRACT

Disclosed is an electronic device for monitoring a voice and laryngeal disorders, which includes a substrate, a first pressure sensor array disposed along a first direction on the substrate and including a plurality of first pressure sensors each extending in a second direction different from the first direction, and at least one second pressure sensor extending in the first direction on the substrate and disposed to be spaced apart from the first pressure sensor array in the second direction. The voice and laryngeal disorder of a user equipped with the electronic device according to the present disclosure may be simultaneously monitored.

### CROSS-REFERENCE TO RELATED APPLICATIONS

This application claims priority under 35 U.S.C. § 119 to Korean Patent Application No. 10-2021-0154106, filed on Nov. 10, 2021, in the Korean Intellectual Property Office, the disclosures of which are incorporated by reference herein in their entireties.

### BACKGROUND

Embodiments of the present disclosure described herein relate to an electronic device for monitoring voice and laryngeal disorders, and more particularly, relate to an electronic device for monitoring voice and laryngeal disorders, which provides electrical signals corresponding to a voice variation of a user and a larynx movement of the user by using a pressure sensor.

The number of patients with voice and laryngeal disorders is increasing every year, and medical expenses are continuously increasing due to this. After diagnosis of the voice and laryngeal disorders, treatment methods include a voice therapy, a drug therapy, or a surgery. In particular, during the rehabilitation process after the surgery, it is absolutely necessary to check the progress by simultaneously observing vocalization, breathing, and swallowing. However, the diagnosis and observation of the voice and laryngeal disorders are currently performed through a physical examination by a doctor. Other than that, the diagnosis and observation of the voice and laryngeal disorders may use implantable devices or indirect imaging methods. However, the implantable devices may be objectionable to patients, and the indirect imaging methods have a risk of exposure to radiation and difficulty in real-time measurement.

To overcome such issues, technologies for measuring the intensity of a vocalization and a voice using a microphone in relation to the voice disorders is proposed. However, when the microphone is used, noise is generated depending on an external environment, such as a noise sound, and thus a signal for measurement may be distorted. In addition, since movement of the larynx cannot be simultaneously sensed during measuring, there are issues in that voice and laryngeal disorders cannot be comprehensively observed.

### SUMMARY

Embodiments of the present disclosure provide an electronic device that allows user's voice and laryngeal disorders to be simultaneously monitored, by providing electrical signals corresponding to a voice variation of a user and a larynx movement of the user, using a pressure sensor.

According to an embodiment of the present disclosure, an electronic device for monitoring voice and laryngeal disorders, which includes a substrate, a first pressure sensor array disposed along a first direction on the substrate and including a plurality of first pressure sensors each extending in a second direction different from the first direction, and at least one second pressure sensor extending in the first direction on the substrate and disposed to be spaced apart from the first pressure sensor array in the second direction.

According to an embodiment of the present disclosure, an electronic device for monitoring voice and laryngeal disorders, which includes a substrate, and a first pressure sensor and a second pressure sensor on the substrate, and the first pressure sensor senses a first pressure and generates a first electrical signal, the second pressure sensor senses a second pressure and generates a second electrical signal, the first electrical signal corresponds to a pressure change caused by a movement of a larynx, and the second electrical signal corresponds to a pressure change caused by a vibration of the voice.

### BRIEF DESCRIPTION OF THE FIGURES

The above and other objects and features of the present disclosure will become apparent by describing in detail embodiments thereof with reference to the accompanying drawings.

FIG. 1 is a diagram illustrating an electronic device according to an embodiment of the present disclosure.

FIG. 2 is a cross-sectional view of an electronic device taken along a line A-A′ of FIG. 1.

FIG. 3 is a diagram illustrating an electronic device according to an embodiment of the present disclosure.

FIG. 4 is a schematic diagram illustrating an electronic device according to an embodiment of the present disclosure.

FIG. 5 is a schematic diagram illustrating an example in which an electronic device of FIG. 4 is attached to a user's neck skin.

FIGS. 6A to 6B are graphs illustrating an operation of a second pressure sensor, according to an embodiment of the present disclosure.

FIG. 7 is a graph illustrating an operation of a second pressure sensor, according to an embodiment of the present disclosure.

FIGS. 8A to 8B are graphs illustrating an operation of a first pressure sensor array, according to an embodiment of the present disclosure.

FIGS. 9A to 9B are graphs illustrating an operations of a first pressure sensor and a second pressure sensor in the same time period, according to an embodiment of the present disclosure.

### DETAILED DESCRIPTION

Hereinafter, embodiments of the present disclosure will be described clearly and in detail such that those skilled in the art may easily carry out the present disclosure.

Further, embodiments described herein will be described with reference to cross-sectional and/or plan views, which are ideal illustrative views of the present disclosure. In drawings, the thicknesses of the layers and regions may be exaggerated to describe the technical features effectively. As such, variations from the shapes of the illustrations as a result, for example, of manufacturing techniques and/or tolerances, are to be expected. Thus, embodiments of the present disclosure should not be construed as limited to the particular shapes illustrated herein but are to include deviations in shapes that result, for example, from manufacturing. For example, the etched region illustrated at a right angle may be rounded or have a predetermined curvature. Thus, the regions illustrated in the drawings are schematic in nature and their shapes are intended to illustrate the specific shapes of the regions of the device and are not intended to limit the scope of the present disclosure.

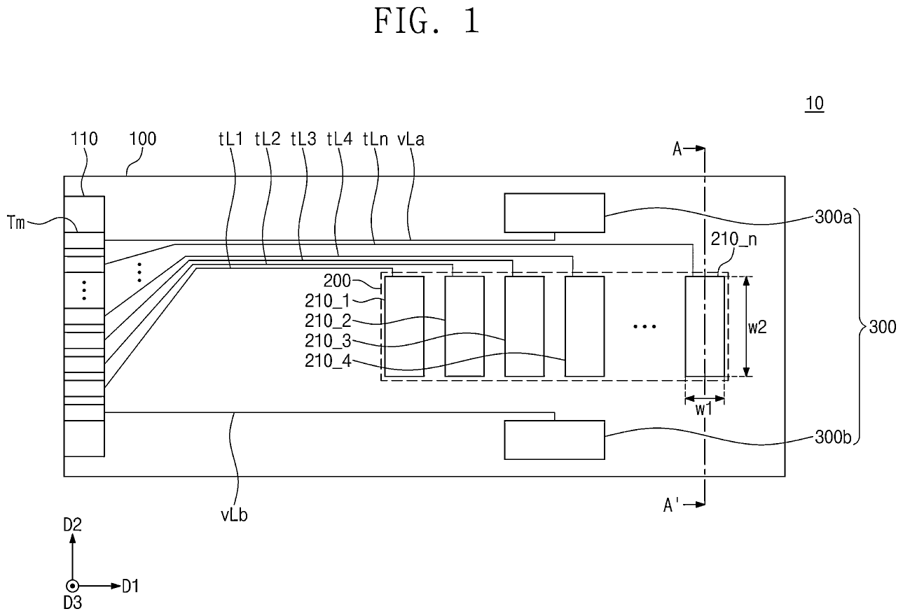

FIG. 1 illustrates an electronic device according to an embodiment of the present disclosure. An electronic device 10 may be attached to a user's neck skin. For example, the electronic device 10 may be attached to the skin around the user's larynx. According to an embodiment of the present disclosure, the electronic device 10 may generate electrical signals using a plurality of pressure sensors. For example, the electrical signal may be a signal corresponding to a variation in the user's voice and/or a larynx movement of the user. The electronic device 10 may transfer an electrical signal to an externally provided display device (not illustrated). Referring to FIG. 1, the electronic device 10 may include a substrate 100, a first pressure sensor array 200, and a second pressure sensor 300.

The substrate 100 may include a terminal unit 110 and wirings tL1 to tLn, vLa, and vLb. For example, the substrate 100 may include glass, plastic, an organic material, and silicon. For example, the substrate 100 may include a printed circuit board (PCB) and/or a flexible printed circuit board (F-PCB).

The terminal unit 110 may include a plurality of terminals ‘Tm’ for outputting a signal to the outside. The plurality of terminals Tm may be exposed to the outside, and may be subjected to surface treatment such as plating to prevent discoloration or surface oxidation. The wirings tL1 to tLn, vLa, and vLb may extend in a first direction D1. The wirings tL1 to tLn, vLa, and vLb form a path for transferring output signals from first pressure sensors 210_1 to 210_n and second pressure sensors 300a and 300b to the terminal unit 110. The terminal unit 110 may be provided on one side of a first surface of the substrate 100.

The first pressure sensor array 200 may be provided on the substrate 100. The first pressure sensor array 200 may include the plurality of first pressure sensors 210_1 to 210_n. The number of the first pressure sensors 210_1 to 210_n may be ‘n’. Here, ‘n’ is any natural number. Each of the first pressure sensors 210_1 to 210_n may extend in a second direction D2 different from the first direction D1. For example, the first direction D1 and the second direction D2 may be perpendicular to each other. The first pressure sensors 210_1 to 210_n may be arranged to be spaced apart from each other on the first surface of the substrate 100 in the first direction D1. A distance between two adjacent pressure sensors among the first pressure sensors 210_1 to 210_n may be uniform. As an example, each of the first pressure sensors 210_1 to 210_n may be arranged to be spaced apart from each other by a width of 10 mm. However, the present disclosure is not limited thereto. Accordingly, a width at which each of the first pressure sensors 210_1 to 210_n is spaced apart may be different from one another.

Each of the first pressure sensors 210_1 to 210_n may have a first width w1 in the first direction D1 and a second width w2 in the second direction D2. For example, the first pressure sensors 210_1 to 210_n may have the same first width w1 and the same second width w2. Accordingly, each of the first pressure sensors 210_1 to 210_n may have the same area. However, the present disclosure is not limited thereto. According to an embodiment of the present disclosure, the first pressure sensors 210_1 to 210_n may have the different first width w1 and/or the different second width w2. For example, as the first pressure sensors 210_1 to 210_n close to one side of the first surface of the substrate 100 along the first direction D1, the first width w1 may increase and the second width w2 may decrease. That is, the size of each of the first pressure sensors 210_1 to 210_n may be the same or different. For example, the size of each of the first pressure sensors 210_1 to 210_n may gradually increase or decrease in the first direction D1, and accordingly, each of the first pressure sensors 210_1 to 210_n may have a different size.

Each of the first pressure sensors 210_1 to 210_n may have a rectangular shape. However, the present disclosure is not limited thereto. Accordingly, each of the first pressure sensors 210_1 to 210_n may be changed to a shape other than the rectangle. For example, each of the first pressure sensors 210_1 to 210_n may have any other shape such as a square shape, a rectangular shape, a circular shape, an oval shape, a triangular shape, a trapezoidal shape, or an irregular shape.

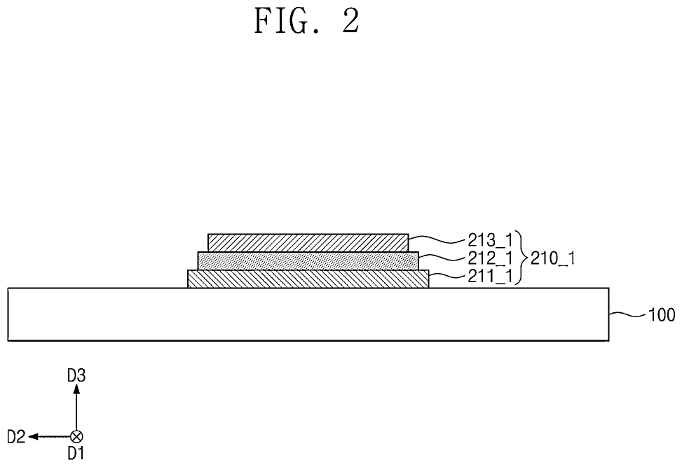

Each of the first pressure sensors 210_1 to 210_n may be electrically connected to each of the wirings tL1 to tLn. For example, the first pressure sensor 210_1 may be electrically connected to the wiring tL1. Although each of the first pressure sensors 210_1 to 210_n is illustrated as being connected to one wiring, each of the first pressure sensors 210_1 to 210_n may be connected to a pair of wirings. A connection relationship between the first pressure sensors 210_1 to 210_n and the wirings tL1 to tLn will be described in detail with reference to FIG. 2.

The at least one second pressure sensor 300 may be provided on the substrate 100. For example, it is illustrated that two second pressure sensors 300a and 300b are provided on the substrate 100. Each of the two second pressure sensors 300a and 300b may extend in the first direction D1 and may be arranged along the second direction D2 with the first pressure sensor array 200 interposed therebetween.

As an example, the one 300a of the second pressure sensors may be disposed to be spaced apart from the first pressure sensor array 200 in the second direction D2, and the other one 300b of the second pressure sensors may be disposed to be spaced apart from the first pressure sensor array 200 in a direction opposite to the second direction D2. FIG. 1 illustrates that the two second pressure sensors 300a and 300b are provided on the substrate 100, but the number and arrangement direction of the second pressure sensor 300 are not limited thereto.

Each of the second pressure sensors 300a and 300b may be electrically connected to each of the wirings vLa and vLb. For example, the one 300a of the second pressure sensors may be electrically connected to the wiring vLa, and the other one 300b of the second pressure sensors may be electrically connected to the wiring vLb. Although each of the second pressure sensors 300a and 300b is illustrated as being connected to one wiring, each of the second pressure sensors 300a and 300b may be connected to a pair of wirings.

The shape of the second pressure sensor 300 may be the same as that of the first pressure sensors 210_1 to 210_n. Accordingly, the second pressure sensor 300 may have a rectangular shape. However, the present disclosure is not limited thereto. The shape of the second pressure sensor 300 may be different from that of the first pressure sensors 210_1 to 210_n. In addition, a shape of the second pressure sensor 300 may have any other shape, such as a square shape, a rectangular shape, a circular shape, an oval shape, a triangular shape, a trapezoidal shape, or an irregular shape.

The first pressure sensors 210_1 to 210_n and the second pressure sensor 300 may sense an external stimulus and may generate a sensing signal based on the sensed external stimulus. For example, the first pressure sensors 210_1 to 210_n and the second pressure sensor 300 may be implemented with a piezoelectric device that senses a pressure in a specific direction and generates a sensing signal proportional to the sensed pressure.

Although not illustrated, the electronic device 10 may communicate with an external device by wiredly and/or wirelessly. In detail, the electronic device 10 may include a communication unit (not illustrated) or an interface (not illustrated) for communication.

The communication unit or the interface for communication may perform communication based on at least one of various wireless communication methods, such as an LTE (Long Term Evolution), a WiMax, a GSM (Global System for Mobile communication), a CDMA (Code Division Multiple Access), a Bluetooth, an NFC (Near Field Communication), a Wi-Fi, and an RFID (Radio Frequency IDentification), or at least one of various wired communication methods, such as a USB (Universal Serial Bus), a SATA (Serial AT Attachment), an SCSI (Small Computer Small Interface), a Firewire, and a PCI (Peripheral Component Interconnection).

FIG. 2 is a cross-sectional view of an electronic device taken along a line A-A′ of FIG. 1. Referring to FIG. 2 together with FIG. 1, the first pressure sensor 210_1 may include a first electrode 211_1, a piezoelectric polymer layer 212_1, and a second electrode 213_1. The first electrode 211_1, the piezoelectric polymer layer 212_1, and the second electrode 213_1 may be disposed on the substrate 100 along a third direction D3 perpendicular to the first and second directions D1 and D2.

The first electrode 211_1 may be disposed on the substrate 100. However, the present disclosure is not limited thereto. For example, the first electrode 211_1 may be included in the substrate 100. The piezoelectric polymer layer 212_1 may be disposed on the first electrode 211_1. The second electrode 213_1 may be disposed on the piezoelectric polymer layer 212_1.

Although not illustrated, the first pressure sensor 210_1 may further include an insulating layer (not illustrated). The insulating layer (not illustrated) may insulate the second electrode 213_1 from the first electrode 211_1. For example, the insulating layer (not illustrated) may be provided between the first electrode 211_1 and the piezoelectric polymer layer 212_1. As another example, the insulating layer (not illustrated) may be provided between the second electrode 213_1 and the piezoelectric polymer layer 212_1.

According to an embodiment of the present disclosure, the piezoelectric polymer layer 212_1 may include at least one of epoxy, silicone rubber, polymethylmethacrylate (PMMA), polyurethane, polydimethyl siloxane (PDMS), polyvinylidenefluoride (PVDF), poly(vinylidenefluoride-co-trifluoroethylene) (P(VDF-TrFE)), PZT, PLZT, PZN-PT, PMN-PT, PIN-PT, PZN-PNN-PZT, BNT, AlN, ZnO, and KNN.

According to an embodiment of the present disclosure, the thickness of the piezoelectric polymer layer 212_1 may be 0.1 to 1000 μm. Although FIG. 2 illustrates that the piezoelectric polymer layer 212_1 is formed as a single layer, the present disclosure is not limited thereto. For example, the piezoelectric polymer layer 212_1 may be stacked in two or more layers.

The wiring tL1 (refer to FIG. 1) may include a pair of wirings. In this case, the one of the pair of wirings (e.g., tL1 of FIG. 1) may be connected to the first electrode 211_1 of the first pressure sensor 210_1, and the other one may be connected to the second electrode 213_1 of the first pressure sensor 210_1. Although not illustrated, the substrate 100 may include additional wiring (not illustrated). The additional wiring (not illustrated) may be electrically connected to the first pressure sensor 210_1.

FIG. 2 illustrates only one (i.e., the first pressure sensor 210_1) of the first pressure sensors 210_1 to 210_n, but each of the remaining first pressure sensors 210_2 to 210_n may have a structure similar to that of the first pressure sensor 210_1 illustrated in FIG. 2. Also, the second pressure sensor 300 may have a structure similar to that of the first pressure sensor 210_1 illustrated in FIG. 2.

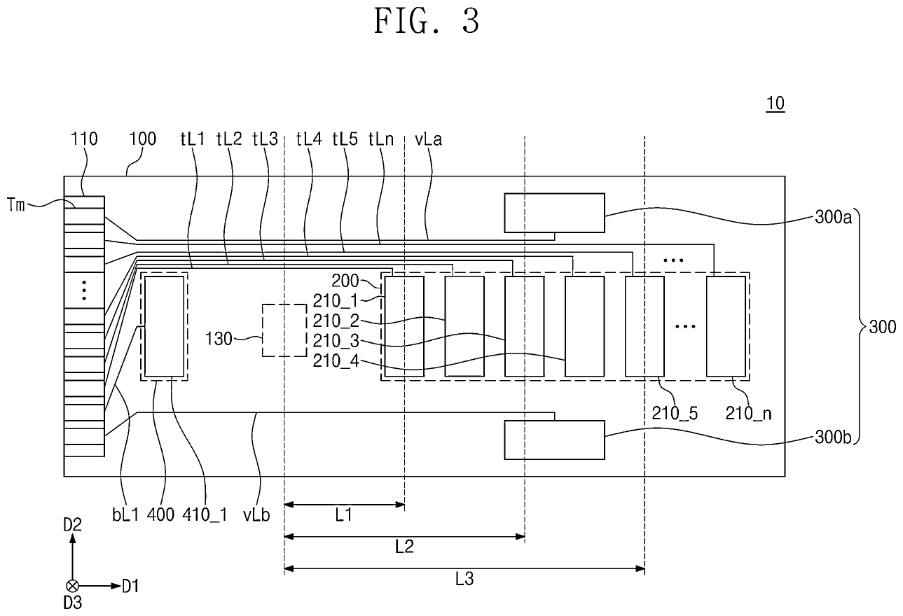

FIG. 3 is a diagram illustrating an electronic device according to an embodiment of the present disclosure. According to an embodiment of the present disclosure, the electronic device 10 may further include a third pressure sensor array 400. The arrangement, connection relationship, and function of the substrate 100, the first pressure sensor array 200, and the second pressure sensor 300 are similar to those described with reference to FIG. 1, and thus additional descriptions thereof will be omitted to avoid redundancy.

The third pressure sensor array 400 may be provided on the substrate 100. The third pressure sensor array 400 may include a third pressure sensor 410_1. The third pressure sensor array 400 may be disposed to be spaced apart from the first pressure sensor array 200 along the first direction D1 on the substrate 100.

For example, it is illustrated that the one third pressure sensor 410_1 is provided on the substrate 100. However, the present disclosure is not limited thereto. The third pressure sensor array 400 may include a plurality of third pressure sensors.

In this case, the plurality of third pressure sensors may be arranged to be spaced apart from one another in the first direction D1. A width at which each of the plurality of third pressure sensors is spaced apart may be uniform. Each of the plurality of third pressure sensors may have the same size. Each of the plurality of third pressure sensors may have a rectangular shape. However, the present disclosure is not limited thereto. Accordingly, a size of the width at which each of the plurality of third pressure sensors is spaced apart from one another and a size and shape of each of the plurality of third pressure sensors may be different from one another.

A wiring bL1 may form a path for transferring an output signal from the third pressure sensor 410_1 to the terminal unit 110. The third pressure sensor 410_1 may be electrically connected to the wiring bL1. Although the third pressure sensor 410_1 is illustrated as being connected to one wiring, each of the third pressure sensors 410_1 may be connected to a pair of wirings.

Referring to FIG. 3 together with FIG. 2, the third pressure sensor 410_1 may have a structure similar to that of the first pressure sensor 210_1 illustrated in FIG. 2.

According to an embodiment of the present disclosure, the substrate 100 may further include a laryngeal attachment region 130. The laryngeal attachment region 130 may be a region between the first pressure sensor array 200 and the third pressure sensor array 400 on the first surface of the substrate 100. According to an embodiment of the present disclosure, the first pressure sensor array 200 may be disposed to be spaced apart from the laryngeal attachment region 130 by 0 to 5 cm.

A center of the laryngeal attachment region 130 may be spaced apart from the center of the first pressure sensor 210_1 by a first distance L1. For example, the first distance L1 may be 10 mm. The center of the laryngeal attachment region 130 may be spaced apart from the center of the first pressure sensor 210_3 by a second distance L2. For example, the second distance L2 may be 20 mm. The center of the laryngeal attachment region 130 may be spaced apart from the center of the first pressure sensor 210_5 by a third distance L3. For example, the third distance L3 may be 30 mm.

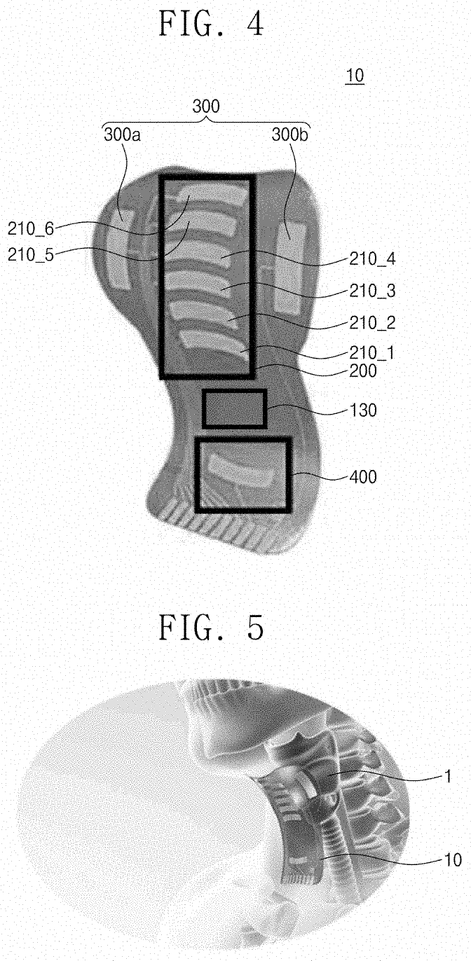

FIG. 4 is a schematic diagram illustrating an electronic device according to an embodiment of the present disclosure. Contents that overlap with those described in FIGS. 1 to 3 will be omitted to avoid redundancy. Along with FIGS. 1 to 3 and referring to FIG. 4, the first pressure sensor array 200, the second pressure sensor 300, and the third pressure sensor array 400 may be disposed on the first surface of the substrate 100. The first pressure sensor array 200 may include the six first pressure sensors 210_1 to 210_6. The third pressure sensor array 400 may include the one third pressure sensor 410_1.

The first pressure sensor array 200 may sense a movement of the larynx of the user. The first pressure sensor array 200 may include the plurality of first pressure sensors 210_1 to 210_6 arranged along the direction of the larynx (e.g., the first direction D1) to sense a pressure change that occurs when the larynx passes through a specific position. Each of the plurality of first pressure sensors 210_1 to 210_6 may output an electrical signal corresponding to the sensed pressure change.

The second pressure sensor 300 may sense a user's voice. The second pressure sensor 300 may sense a vibration of the skin of the neck caused by the user's voice or a specific situation (e.g., coughing, sneezing, etc.). That is, the second pressure sensor 300 may sense a pressure change that occurs when the skin of the neck is vibrated. Since the second pressure sensor 300 senses the user's voice using the vibration, noise caused by external noise may not occur in an output of the second pressure sensor 300. In addition, even if the volume of the user's voice is small due to a specific situation (e.g., a situation in which the user wears a mask), the second pressure sensor 300 may accurately sense the voice or the like. The second pressure sensor 300 may output the electrical signal corresponding to the sensed pressure change. According to an embodiment of the present disclosure, the second pressure sensor 300 may sense a frequency of 50 to 3000 Hz.

The third pressure sensor array 400 may sense a movement of the cricoid cartilage of the user. The third pressure sensor array 400 may sense a pressure change that occurs when the cricoid cartilage passes through a specific position. According to an embodiment of the present disclosure, the third pressure sensor array 400 may include the third pressure sensor 410_1. The third pressure sensor 410_1 may output the electrical signal corresponding to the sensed pressure change.

The electronic device 10 may output electrical signals from the plurality of first pressure sensors 210_1 to 210_6, the second pressure sensor 300, and the third pressure sensor 410_1. The electrical signals are simultaneously monitored, such that a user's voice and a user's laryngeal disorder may be managed or diagnosed in real time.

FIG. 5 is a schematic diagram illustrating an example in which an electronic device of FIG. 4 is attached to a user's neck skin. Contents that overlap with those described in FIGS. 1 to 4 will be omitted to avoid redundancy. Referring to FIG. 5, together with FIG. 4, the first pressure sensor array 200 may be attached to the skin of the neck corresponding to a larynx 1 of the user. The first pressure sensor array 200 may sense a movement of the larynx 1 caused by a specific situation (e.g., swallowing saliva, swallowing water, etc.) through a pressure change on the skin of the neck.

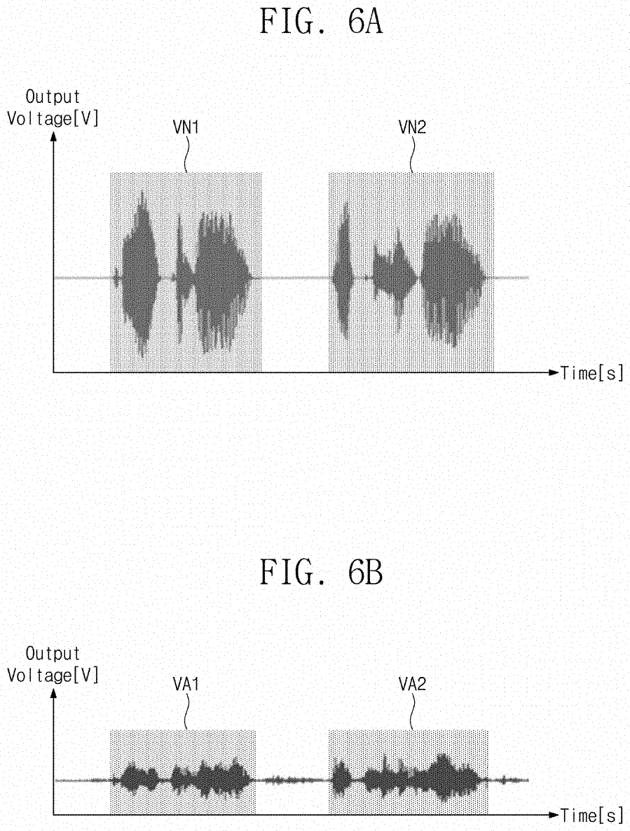

FIGS. 6A to 6B are graphs illustrating an operation of a second pressure sensor, according to an embodiment of the present disclosure. Referring to FIGS. 6A to 6B, together with FIGS. 1 to 5, an x-axis represents a time, and a y-axis represents a level of an output voltage of the second pressure sensor 300. In this case, the level of the output voltage of the second pressure sensor 300 may correspond to a voltage level of an electrical signal output by the second pressure sensor 300.

Referring to FIG. 6A, a first normal voice period VN1 and a second normal voice period VN2 are time periods when the user wearing the electronic device 10 utters an example sentence with a voice in a normal state. In this case, the example sentence in the first normal voice period VN1 is “Open the door”, and the example sentence in the second normal voice period VN2 is “Close the door”.

Referring to FIG. 6B, a first abnormal voice period VA1 and a second abnormal voice period VA2 are time periods when the user wearing the electronic device 10 utters an example sentence with a voice in an abnormal state (e.g., a state in which the voice is hoarse). In this case, the example sentence in the first abnormal voice period VA1 is “Open the door”, and the example sentence in the second abnormal voice period VA2 is “Close the door”.

The first normal voice period VN1 and the first abnormal voice period VA1 correspond to each other, and the second normal voice period VN2 and the second abnormal voice period VA2 correspond to each other. The waveforms in the first normal voice period VN1 and the first abnormal voice period VA1 represent that a change between the output voltages occurs at the same or similar time points. However, the amplitude of the waveform in the first abnormal voice period VA1 is relatively small compared to the amplitude of the waveform in the first normal voice period VN1. As in the above description, the waveforms in the second normal voice period VN2 and the second abnormal voice period VA2 represent that a change between the output voltages occurs at the same or similar time points. However, the amplitude of the waveform in the second abnormal voice period VA2 is relatively small compared to the amplitude of the waveform in the second normal voice period VN2. Accordingly, the user's voice disorder may be determined by monitoring the waveforms in each time period.

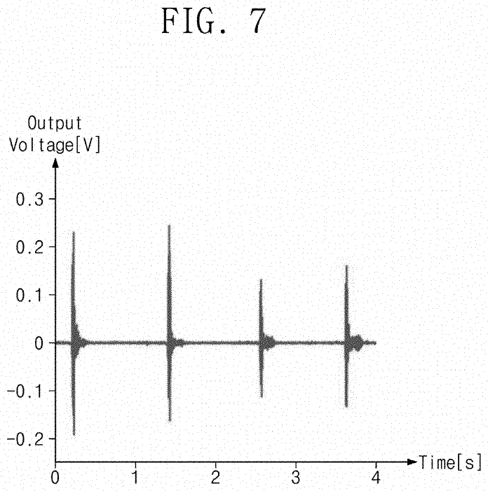

FIG. 7 is a graph illustrating an operation of a second pressure sensor, according to an embodiment of the present disclosure. Referring to FIG. 7, together with FIGS. 1 to 5, an x-axis represents a time, and a y-axis represents a level of an output voltage of the second pressure sensor 300. In this case, a level of the output voltage of the second pressure sensor 300 may correspond to the voltage level of the electrical signal output from the second pressure sensor 300 when the user wearing the electronic device 10 coughs. When the user coughs, the second pressure sensor 300 may momentarily output an electrical signal having a large voltage level.

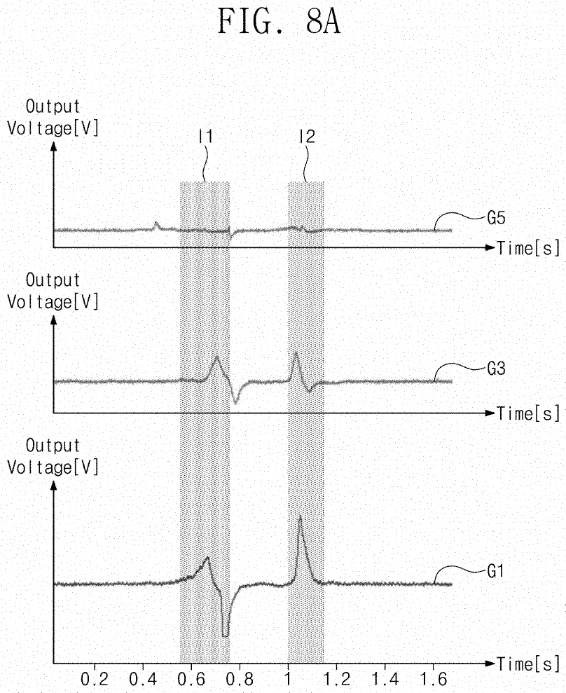

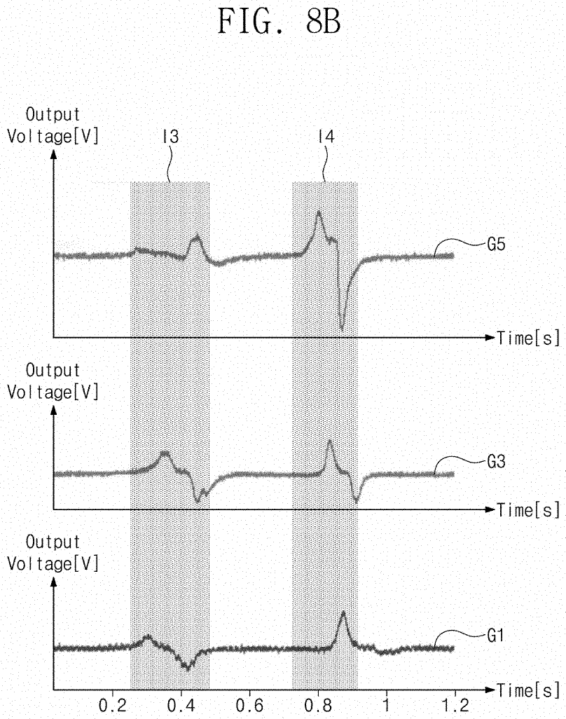

FIGS. 8A to 8B are graphs illustrating an operation of a first pressure sensor array, according to an embodiment of the present disclosure. With reference to FIGS. 8A to 8B, together with FIG. 3, an x-axis represents a time, and a y-axis represents a level of the output voltage of the first pressure sensors 210_1, 210_3, and 210_5. In this case, levels of the output voltages of the first pressure sensors 210_1, 210_3, and 210_5 may correspond to voltage levels of the electric signals output from the first pressure sensors 210_1, 210_3, and 210_5, respectively.

The first pressure sensor array 200 may include the ‘n’ first pressure sensors 210_1 to 210_n. The ‘n’ first pressure sensors 210_1 to 210_n may correspond to first to n-th channels of the first pressure sensor array 200, respectively. For example, the first pressure sensor 210_3 may correspond to the third channel of the first pressure sensor array 200. For convenience of description, output voltage changes in the first channel, the third channel, and the fifth channel will be described below.

Referring to FIG. 8A, a user wearing the electronic device 10 may perform a saliva swallowing operation. In this case, the first pressure sensors 210_1 to 210_n may sense a movement of the larynx by a saliva swallowing operation. That is, the first pressure sensors 210_1 to 210_n may output an electrical signal corresponding to a pressure change depending on the movement of the larynx.

A first period I1 is a time period corresponding to a laryngeal raising operation, and a second period I2 is a time period corresponding to a laryngeal falling operation. In the first period I1 and the second period I2, the first channel disposed closest to the laryngeal attachment region 130 may first sense the movement of the larynx to output an electrical signal G1. In turn, the third channel may output an electrical signal G3 by sensing the movement of the larynx. However, referring to a waveform of an electrical signal G5, the fifth channel disposed relatively far from the laryngeal attachment region 130 hardly senses the vertical movement of the larynx due to the saliva swallowing operation.

Referring to FIG. 8B, a user wearing the electronic device 10 may perform a water swallowing operation. In this case, the first pressure sensors 210_1 to 210_6 may sense the movement of the larynx by the water swallowing operation. That is, the first pressure sensors 210_1 to 210_n may output an electrical signal corresponding to a pressure change depending on the movement of the larynx.

A third period I3 is a time period corresponding to a laryngeal raising operation, and a fourth period I4 is a time period corresponding to a laryngeal falling operation. In the third period I3 and the fourth period I4, the first channel disposed closest to the laryngeal attachment region 130 may first sense the movement of the larynx to output the electrical signal G1. In turn, the third channel and the fifth channel may output electrical signals G3 and G5 by sensing the movement of the larynx. Since the vertical movement of the larynx is larger in the water swallowing operation in FIG. 8B compared to the saliva swallowing operation in FIG. 8A, the fifth channel may also sense the movement of the larynx.

Referring to FIGS. 8A to 8B, the position of the larynx may be monitored using the time of change of the output voltages generated in the first to fourth periods I1 to I4, and the rising and falling speeds of the larynx may be calculated. A user's dysphagia may be determined using the position of the larynx and the rising and fall speeds of the larynx.

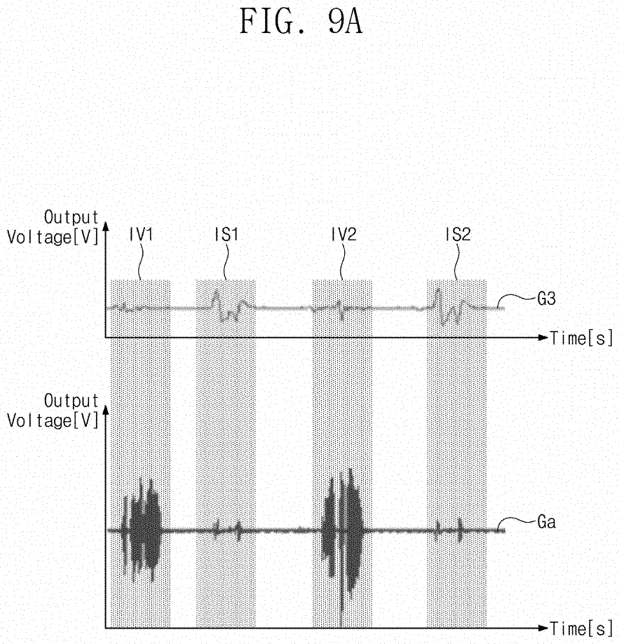

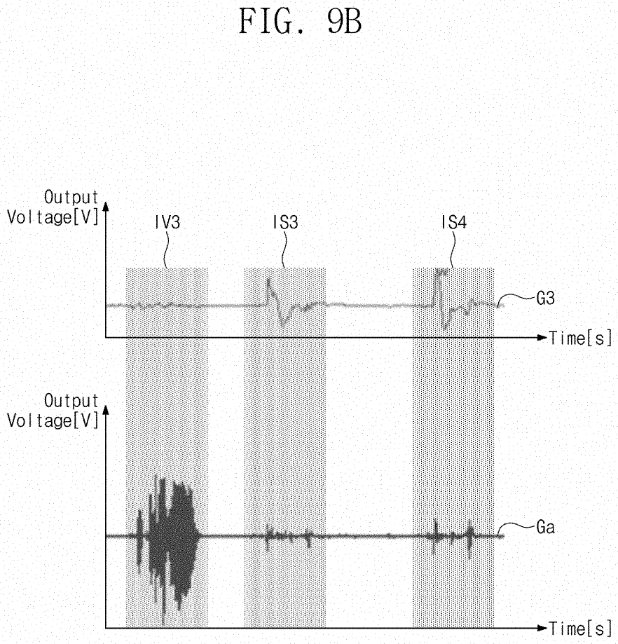

FIGS. 9A to 9B are graphs illustrating an operations of a first pressure sensor and a second pressure sensor in the same time period, according to an embodiment of the present disclosure. Referring to FIGS. 9A to 9B, together with FIG. 4, an x-axis represents a time, and a y-axis represents a level of the output voltage of the first pressure sensor 210_4 and a level of the output voltage of the second pressure sensor 300a. In this case, a level of an output voltage of the first pressure sensor 210_4 may be a voltage level of an electrical signal G4 output from the first pressure sensor 210_4, and a level of an output voltage of the second pressure sensor 300a may be a voltage level of an electrical signal Ga output from the second pressure sensor 300a. For convenience of description, although illustrated in relation to the operations of the first pressure sensor 210_4 and the second pressure sensor 300a, the present disclosure is not limited thereto. For example, operations of the remaining first pressure sensors 210_1, 210_2, 210_3, 210_5, and 210_6 and the remaining second pressure sensor 300b may be similar to the operations of the first pressure sensor 210_4 and the second pressure sensor 300a.

Referring to FIGS. 9A to 9B, first to third voice periods IV1 to IV3 are time periods when a user wearing the electronic device 10 utters an example sentence with a voice. In this case, the example sentence in the first voice period IV1 and the third voice period IV3 is “Open the door”, and the example sentence in the second voice period IV2 is “Close the door”. First to fourth swallowing periods IS1 to IS4 are time periods corresponding to movements of the larynx when the user wearing the electronic device 10 performs a swallowing operation.

In the first to third voice periods IV1 to IV3, the second pressure sensor 300a may sense the user's voice and may output the electrical signal Ga. In contrast, in the first to third voice periods IV1 to IV3, it may be seen that the waveform of the electric signal G4 output from the first pressure sensor 210_4 has no change compared to the waveform of the electric signal Ga. That is, even if the user wearing the electronic device 10 makes a voice, this may not affect functions of the second pressure sensor 210_4 sensing the user's swallowing operation.

In addition, in the first to fourth swallowing periods IS1 to IS4, the first pressure sensor 210_4 may sense the user's laryngeal movement and may output the electrical signal G4. In contrast, in the first to fourth swallowing periods IS1 to IS4, it may be seen that the waveform of the electric signal Ga output from the second pressure sensor 300a has no change compared to the waveform of the electric signal G4. That is, even if the user wearing the electronic device 10 performs the swallowing operation, this may not affect functions of the first pressure sensor 300a sensing the user's voice.

Referring to FIGS. 9A to 9B, together with FIGS. 1 and 4, the operation of sensing the movement of the user's larynx by the first pressure sensor 210_4 and the operation of sensing the user's voice by the second pressure sensor 300a may be performed without an interference with each other. Since this is only an example, the present disclosure is not limited thereto, and all the first pressure sensors 210_1 to 210_n and all the second pressure sensors 300a and 300b of the electronic device 10 may also operate without mutual interference. Since the first pressure sensors 210_1 to 210_n and the second pressure sensors 300a and 300b of the electronic device 10 may operate without interference with one another, the user's voice and laryngeal disorders may be simultaneously monitored through the electronic device 10.

According to an embodiment of the present disclosure, the voice and laryngeal disorders of a user equipped with an electronic device may be simultaneously monitored.

According to an embodiment of the present disclosure, by using a soft material with respect to the substrate and the pressure sensor, it is possible to improve the wearing comfort of the user who attaches the electronic device.

According to an embodiment of the present disclosure, since a user's voice is sensed using a pressure sensor, accurate detection is possible regardless of external environments such as surrounding noise.

The above description refers to embodiments for implementing the present disclosure. Embodiments in which a design is changed simply or which are easily changed may be included in the present disclosure as well as an embodiment described above. In addition, technologies that are easily changed and implemented by using the above embodiments may be included in the present disclosure. While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. An electronic device for monitoring voice and laryngeal disorders, the electronic device comprising:
a substrate;
a first pressure sensor array disposed along a first direction on the substrate and including a plurality of first pressure sensors each extending in a second direction different from the first direction; and
at least one second pressure sensor extending in the first direction on the substrate and disposed to be spaced apart from the first pressure sensor array in the second direction.

2. The electronic device of claim 1, further comprising:
a third pressure sensor array disposed to be spaced apart from the first pressure sensor array in the first direction.

3. The electronic device of claim 1, wherein each of the plurality of first pressure sensors has a first width in the first direction and a second width in the second direction, and
wherein each of the plurality of first pressure sensors has the same first width and the same second width.

4. The electronic device of claim 1, wherein each of the plurality of first pressure sensors has a first width in the first direction and a second width in the second direction, and
wherein, as the plurality of first pressure sensors close to one side of the substrate along the first direction, the first width increases and the second width decreases.

5. The electronic device of claim 1, wherein at least one of each of the plurality of first pressure sensors and the second pressure sensor includes:
a first electrode disposed on the substrate;
a piezoelectric polymer layer disposed on the first electrode; and
a second electrode disposed on the piezoelectric polymer layer.

6. The electronic device of claim 5, wherein the piezoelectric polymer layer includes at least one of epoxy, silicone rubber, polymethylmethacrylate (PMMA), polyurethane, polydimethyl siloxane (PDMS), polyvinylidenefluoride (PVDF), poly(vinylidenefluoride-co-trifluoroethylene) (P(VDF-TrFE)), PZT, PLZT, PZN-PT, PMN-PT, PIN-PT, PZN-PNN-PZT, BNT, AlN, ZnO, and KNN.

7. The electronic device of claim 5, wherein the thickness of the piezoelectric polymer layer is 0.1 to 1000 μm.

8. The electronic device of claim 5, wherein the piezoelectric polymer layer is a single layer or is stacked in two or more layers.

9. The electronic device of claim 5, further comprising:
an insulating layer disposed between the first electrode and the second electrode.

10. The electronic device of claim 1, wherein each of the at least one second pressure sensor has a circular shape, a rectangular shape, or a polygonal shape.

11. An electronic device for monitoring voice and laryngeal disorders, the electronic device comprising:
a substrate; and
a first pressure sensor and a second pressure sensor on the substrate, and
wherein the first pressure sensor senses a first pressure and generates a first electrical signal,
wherein the second pressure sensor senses a second pressure and generates a second electrical signal,
wherein the first electrical signal corresponds to a pressure change caused by a movement of a larynx, and
wherein, the second electrical signal corresponds to a pressure change caused by a vibration of the voice.

12. The electronic device of claim 11, further comprising:
a third pressure sensor on the substrate, and
wherein the third pressure sensor senses a third pressure and generates a third electrical signal, and
wherein the third electrical signal corresponds to a pressure change caused by a movement of a cricoid cartilage.
